(19) United States
(12) Patent Application Publication
(10) Pub. No.: US 2024/0358009 A1
(43) Pub. Date: Oct. 31, 2024

(54) INSECT OVIPOSITION COLLECTOR FORMED BY A STRUCTURE HAVING RAISED EGG DEPOSITION PATTERNS

(71) Applicant: Innovafeed, Nesle (FR)

(72) Inventors: Bastien Oggeri, Paris (FR); Xavier Daussin, Nesle (FR); Gustavo Krapf, Nesle (FR); Emmanuel Mernier, Nesle (FR); Géraldine Schuster, Nesle (FR)

(21) Appl. No.: 18/689,401

(22) PCT Filed: Aug. 22, 2022
(86) PCT No.: PCT/FR2022/051597

§ 371 Date: Mar. 5, 2024

(30) Foreign Application Priority Data
Sep. 6, 2021 (FR) FR2109317

(51) Int. Cl. A01K 67/033

(52) U.S. Cl. CPC A01K 67/033

(57) ABSTRACT

An insect oviposition collector is formed by a structure having raised egg deposition patterns, the lower or upper portion of the structure being configured so as to slide, thereby enabling the collector to be inserted into and removed from an oviposition zone.

### CROSS-REFERENCE TO RELATED APPLICATIONS

This application is a national phase entry under 35 U.S.C. § 371 of International Patent Application PCT/FR2022/051597, filed Aug. 22, 2022, designating the United States of America and published as International Patent Publication WO 2023/031537 A1 on Mar. 9, 2023, which claims the benefit under Article 8 of the Patent Cooperation Treaty of French Patent Application Serial No. FR2109317, filed Sep. 6, 2021.

### TECHNICAL FIELD

The present disclosure relates to the field of industrial farming of insects, in particular, of the black soldier fly, intended, for example, for the production of proteins from insect larvae. It relates more particularly to equipment intended for the reproduction and oviposition (mating) step of the insects, starting with the introduction of the pupae and ending with the collection of the eggs, which are then transferred to another equipment item intended for the hatching step. This reproduction and oviposition step is generally done inside cages allowing insects to have sufficient living space while remaining confined in an enclosed space. The climate conditions are controlled inside these cages in order to guarantee optimal conditions.

### BACKGROUND

Collectors are arranged in these cages in order to encourage insects to lay eggs in selected locations, and to facilitate the large-scale collection of eggs.

French Patent No. FR3027488 is known in the state of the art and relates to an insect farming unit comprising a closed chamber and a system for controlling the environmental conditions of the chamber. This closed chamber comprises an upper compartment for containing insects, with an airlock, used to pass food; the closed chamber also comprises at least one lower compartment for receiving the insect eggs contained in the upper compartment; the closed chamber further comprises at least one separating element for separating the upper and lower compartments.

The oviposition collector is constituted by the lower compartment referenced (5): at least one lower compartment (5) for receiving the eggs of insects contained in the upper compartment (3). The element referenced (7) is a frame “in particular, allowing insects to hide or molt on a support.”

French Patent No. FR3053212 discloses a collecting and harvesting device suitable for the oviposition and harvesting of insect eggs, the harvesting device comprising: supports, superimposed on one another, spacers, each of the spacers being inserted between two adjacent supports, among the superimposed supports, a spacer member movable between a retracted position wherein the supports are in the oviposition position, and an interaction position wherein the supports are in the harvesting position, the movement of the spacing member from the retracted position to the interaction position driving the movement of the supports from the oviposition position to the harvesting position; in the oviposition position, the spacers are in contact with the two respective adjacent supports, and define an oviposition space between the two adjacent supports, suitable for receiving the eggs laid by the insects, and in the harvesting position, each support is spaced apart from an adjacent support by a distance D greater than the height of the spacer.

French Patent No. FR3066360 discloses an insect oviposition plate comprising at least two removable elements each comprising at least one well, the volume of which is configured to receive eggs laid by an insect; the volume being between 80% and 500% of the volume of eggs laid by the predefined flying insect.

The solutions of the prior art are not completely satisfactory because the manipulation of the egg collection support leads to two risks: a risk of flies escaping during the removal of the collectors from the oviposition zone, and a risk of the eggs detaching during the various manipulations of the collection support, such as, for example, during the transfer to the hatching zone.

Many solutions of the prior art are moreover degradable over time: cardboard cells, moisture-sensitive wood chips, etc., which requires them to be changed repeatedly.

Some solutions consist of an assembly of mobile or removable parts, which makes them fragile and reduces their lifetime, given the manipulations to which they are subjected.

Finally, in several solutions of the state of the art, the eggs or the freshly hatched larvae are recovered in the substrate, which makes it difficult to quantify them precisely. However, quantifying the number of eggs or freshly hatched larvae is essential to correctly control the remainder of the rearing process.

### BRIEF SUMMARY

In order to address these drawbacks, the present disclosure relates, in its most general sense, to an oviposition collector for insects formed by a structure having raised egg deposition patterns, a lower or upper portion of the structure being configured so as to slide in a guide, thereby enabling the collector to be inserted into and removed from the oviposition zone.

Preferably, the structure has, on the upper edge, attachment means suitable for sliding in the guide.

According to a first variant, the structure is constituted by a stamped metal sheet having ribs on both sides of a median plane of the stamped metal sheet.

According to a second variant, the structure consists of a plate made of plastic material, in particular, of PVC, having ribs on both sides of a median plane of the plate. Preferably, the collector is monobloc and has attachment means directly machined into the mass complementary to the profile of the guide.

Alternatively, the attachment means consist of a rod penetrating the collector and extended by a head whose cross section is complementary to the profile of the guide.

According to a particular embodiment, the collector according to the present disclosure has, on the side edge, attachment means for the reversible assembly of two adjacent collectors. Preferably, the attachment means between collectors are magnets.

Preferably, the collectors are black in color.

The present disclosure also relates to a hatch for inserting and removing a collector consisting of the assembly of two bent plates each having a slot suitable for inserting and removing an egg collector, and a door for masking the slot. Alternatively, the hatch has elastic lips ensuring a barrier to the exit of flies while allowing the insertion or removal of the collectors, by elastic deformation of these lips during the passage of the collector in the slot provided on the wall of the cage.

The present disclosure also relates to a cage equipped with an opening for inserting and removing a collector as set out above, characterized in that the opening has a hatch consisting of the assembly of two bent plates each having a slot suitable for inserting and removing an egg collector, and a door for masking the slot. The present disclosure also relates to a cage equipped with an opening for inserting and removing a collector as set out above, characterized in that the opening has lips deformable between a configuration for closing the opening and a configuration suitable for inserting and removing an egg collector.

The present disclosure also relates to an equipment item for cleaning collectors intended for collectors according to the present disclosure, characterized in that the equipment comprises at least one pair of drive rollers between which the collector to be cleaned is engaged and then driven by the counter-rotating rotation of the drive rollers of each pair between a pair of brushing rollers with fibers and rotating at a speed different from the speed of rotation of the drive rollers.

### BRIEF DESCRIPTION OF THE DRAWINGS

Other features and advantages will become apparent from the following description of the present disclosure, given only by way of example, referring to the accompanying drawings wherein:

FIG. 1 shows a front view of a collector according to the present disclosure;

FIG. 2 shows a front view of an assembly of collectors according to the present disclosure;

FIG. 3 shows a perspective view of a hatch for introducing and removing an assembly of collectors;

FIG. 4 shows a schematic view of cleaning equipment for collectors according to the present disclosure.

### DETAILED DESCRIPTION

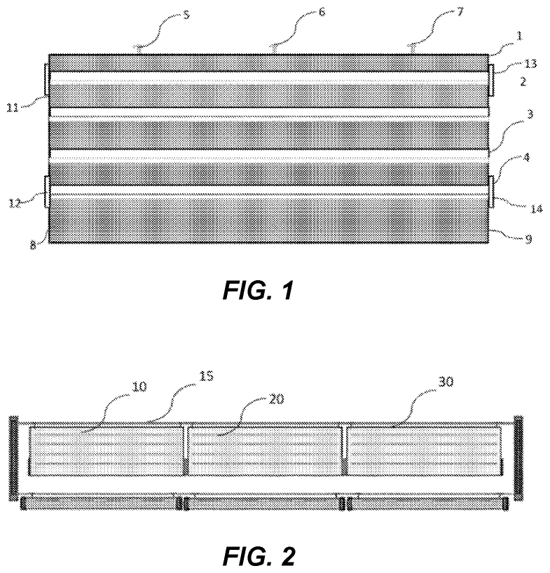

FIG. 1 shows front views of a collector according to the present disclosure and of an assembly of such collectors. A collector (10, 20, 30) consists of a panel, for example, made of a plastic material, of rectangular shape, having ribs (1 to 4). These ribs (1 to 4) form a set of ridges and corners that are conducive to oviposition and holding of the eggs, without allowing insects to be housed therein. The grooves are preferably provided on both sides of the collector in order to increase the oviposition surface. The present disclosure differs from the prior art, in particular, by the fact that oviposition is carried out on these collectors, whereupon they remain attached due to the raised patterns, without falling into a substrate container. These collectors can then be removed from the cage by passing through a vertical slot of the wall of the cage, by a movement in the median plane of the slot by sliding along a direction perpendicular to the plane of the wall, owing to the guides, without losing eggs, to recover them in reliable egg grouping and weighing equipment. Having a reliable quantification of the eggs is essential for monitoring the performance of the reproduction and oviposition process, as well as for the rest of the rearing process, in particular, for assaying the amount of food relative to the amount of eggs or of neonates.

The attachment can be achieved by a protrusion or cavity provided on the upper edge of the collector, cooperating with a complementary means provided in the cage, or a hook extending from the wall.

The collector may consist of a profile where the ribs of one face alternate with the ribs of the opposite face, in order to allow an embodiment by bending of a metal sheet or thermoforming of a plate made of plastic material.

The collector may consist of a block of plastic material, for example, PVC with a thickness of 5 to 30 millimeters, machined to form ribs with a depth of 2 to 15 millimeters and a height of 1 to 20 millimeters.

The collectors are preferably black. Indeed, tests have shown that in the same cage, in symmetrical locations, flies prefer to lay eggs on dark colors rather than light colors, black having the best results. The upper front face has protrusions (5 to 7) with a rod penetrating into the upper edge of the collector and a flared head, for attachment in a rail (15) and sliding longitudinally relative to this rail, having a hollow core and a slot for the passage of the protrusions (5 to 7).

The side flanks (8, 9) have magnets (11 to 14) for keeping the adjacent collectors (10, 20) connected during their movements relative to the rail (15), for insertion or removal. The action exerted on the accessible collector (10) makes it possible to drive the other magnetically coupled collectors.

The assembly of small-sized collectors reversibly attached by magnets has several advantages:
If the collector was in a single segment, then it would be very difficult to handle. This would, for example, require equipment for suitable hatching, and therefore very large equipment. This would also require a significant backward movement to be able to leave the cage and therefore a non-negligible loss of space.
If the collectors were in several segments but not magnetized, then it would be impossible to handle those in the middle of the cage without increasing the risks of flies escaping.

### Example Hatch

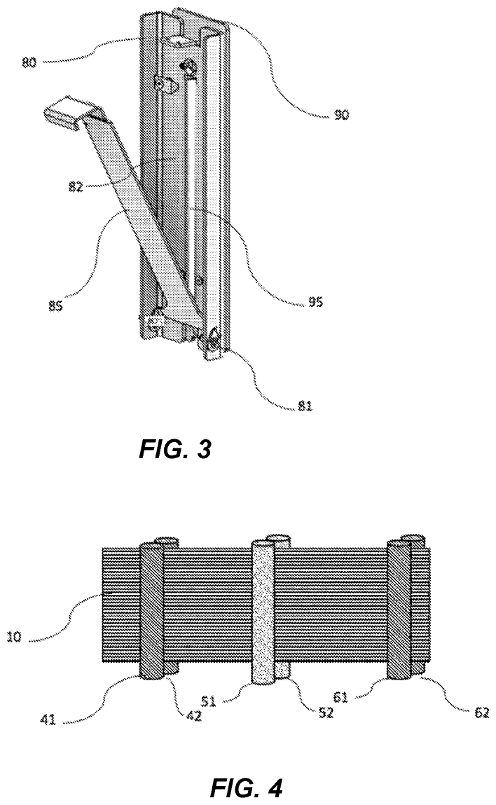

FIG. 3 shows an example of a hatch for inserting and removing egg collectors.

It consists of the assembly of two bent sheets (or plastic parts) (80, 90) each having a slot (95) suitable for inserting and removing an egg collector by sliding along the guides, in a direction perpendicular to the plane of the wall wherein the slot (95) is provided. The slot (95) has a shape and dimensions determined based on the cross section of the egg collector in order to allow it to pass through the slot with a gap preventing the flies in the cage from escaping from the cage.

This slot (95) can be masked by a door (85) tilting about a transverse axis (81). Of course, the hinge can also be provided relative to a vertical axis. A mechanical solution like elastic, magnet, latch (82) ensures that the door (85) is held in a position pressed against the slot (95).

For a flexible cage wall formed by a cloth or netting, the two parts (80; 90) are positioned on both sides of a cutout made in the wall, optionally braided to avoid tearing, and then clamped against one another by screws passing through the two parts (80, 90). The slot made in the netting may be provided with a slide fastener on both sides of the door to allow additional access. The system is also compatible with a zipper and can be positioned on a zipper without the need for an additional cutout. Thus, this also offers positioning flexibility along the zipper.

The assembly is similar for rigid cage walls. In this case, a cutout is made having a dimension greater than that of the slot (95) and less than the cross section of the parts (80, 90) in order to allow them to be positioned on both sides of the wall.

The part (80) can be attached by screwing, for example, on the front end of the guide rails of the egg collectors in order to ensure correct centering.

### Collector Cleaning Equipment

FIG. 4 shows a schematic view of equipment for cleaning ribbed collectors according to the present disclosure.

Cleaning the collectors is an important step in order to be able to reuse the collectors and thus limit the production of waste:
After oviposition, the collectors filled with eggs are brought to the hatching zone.
Once the eggs have hatched, the collectors are recovered to be reused. However, egg residues remain in the slots. Without cleaning, the ribs would gradually become plugged up and would lose their egg retention effectiveness.

However, cleaning inside a rib is not easy, especially taking into account the large number of collectors to be cleaned, hence the need to use a specialized and automatic machine.

According to one example embodiment, this specialized machine consists of two flexible drive rollers (41, 42; 61, 62) between which the collector to be cleaned (10) is engaged and then driven by the counter-rotating rotation of the rollers of each pair between a third pair of brushing rollers (51, 52) with flexible fibers and rotating at a speed different from the rotational speed of the drive rollers (41, 42; 61, 62). They ensure the brushing of the surface of the collector (10) and of the ribs (1 to 4) on both sides of the median plane of the collector (10).

1. An insect oviposition collector comprising a structure having raised egg deposition patterns, a lower or upper portion of the structure being configured so as to slide in a guide, thereby enabling the collector to be inserted into and removed from an oviposition zone.

2. The insect oviposition collector of claim 1, wherein the structure has, on an upper edge, attachment means able to slide in the guide.

3. The insect oviposition collector of claim 1, wherein the structure is constituted by a stamped metal sheet or by a plate made of plastic material having ribs on both sides of the median plane.

4. The insect oviposition collector of claim 2, wherein the collector is monobloc and has attachment means directly machined into a mass complementary to the profile of the guide.

5. The insect oviposition collector of claim 2, wherein the attachment means consist of a rod penetrating the collector and extended by a head whose cross section is complementary to the profile of the guide.

6. The insect oviposition collector of claim 1, wherein it has, on a side edge, attachment means for a reversible assembly of two adjacent collectors.

7. The insect oviposition collector of claim 6, wherein the attachment means are magnets.

8. The insect oviposition collector of claim 1, wherein it is black in color.

9. A hatch for inserting and removing a collector according to claim 1, the hatch comprising an assembly of two bent plates each having a slot suitable for inserting and removing an egg collector, and a door for masking the slot.

10. The hatch of claim 9, further comprising elastic lips ensuring a barrier to an exit of flies while allowing the insertion or removal of the collectors, by elastic deformation of the elastic lips during passage of the collector in a slot provided on a wall of a cage.

11. An oviposition zone equipped with an opening for inserting and removing a collector according to claim 1, wherein the opening has a hatch comprising an assembly of two bent plates each having a slot suitable for inserting and removing an egg collector, and a door for masking the slot.

12. An oviposition zone equipped with an opening for inserting and removing a collector according to claim 1, wherein the opening has lips deformable between a configuration for closing the opening and a configuration suitable for inserting and removing an egg collector.

13. An equipment item for cleaning collectors according to claim 1, the equipment comprising at least one pair of drive rollers between which the collector to be cleaned is engaged and then driven by a counter-rotating rotation of the drive rollers of each pair between a pair of brushing rollers with fibers and rotating at a speed different from the speed of rotation of the drive rollers.
